(19) United States
(12) Patent Application Publication
(10) Pub. No.: US 2011/0130615 A1
(43) Pub. Date: Jun. 2, 2011

(54) MULTI-MODALITY NEUROMODULATION OF BRAIN TARGETS

(76) Inventor: David J. Mishelevich, Playa del Rey, CA (US)

(21) Appl. No.: 12/958,411

(22) Filed: Dec. 2, 2010

Related U.S. Application Data
(60) Provisional application No. 61/266,112, filed on Dec. 2, 2009.

(51) Int. Cl. A61N 2/00; A61N 1/36

(52) U.S. Cl. 600/9; 607/45; 607/3

(57) ABSTRACT

Disclosed are methods and systems and methods for deep or superficial deep-brain stimulation using multiple therapeutic modalities. These impact multiple points in a neural circuit or one or multiple points in multiple neural circuits to produce Long-Term Potentiation (LTP) or Long-Term Depression (LTD) to treat indications such as neurologic and psychiatric conditions. Modality examples are implanted deep-brain stimulators (DBS), Transcranial Magnetic Stimulation (TMS), transcranial Direct Current Stimulation (tDCS), implanted optical stimulation, focused ultrasound, RF stimulation, vagus nerve stimulation, other-implant stimulation, functional stimulation, and drugs. Some targets may be up-regulated and others down-regulated. Coordinated control is provided, as applicable, for control of the direction of the energy emission, intensity, session duration, frequency, pulse-train duration, phase, and numbers of sessions, if and as applicable, for neurormodulation of neural targets. Use of ancillary monitoring or imaging to provide feedback may be applied.

### CROSS REFERENCE TO RELATED APPLICATIONS

This patent application claims priority to provisional patent application 61/266,112, filed Dec. 2, 2009, entitled “MULTI-MODALITY NEUROMODULATION OF BRAIN TARGETS.” The disclosure of this patent application is herein incorporated by reference in its entirety.

### INCORPORATION BY REFERENCE

All publications, including patents and patent applications, mentioned in this specification are herein incorporated by reference in their entirety to the same extent as if each individual publication was specifically and individually indicated to be incorporated by reference.

### FIELD OF THE INVENTION

Described herein are systems and methods for neuromodulation of one or more superficial- or deep-brain targets using more than one means of neuromodulation to up-regulate and/or down-regulate neural activity.

### BACKGROUND OF THE INVENTION

It has been demonstrated that a variety of methods can be employed to neuromodulate superficial or deep brain neural structures. Examples are implanted deep-brain stimulators (DBS), Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, functional stimulation, or drugs. If neural activity is increased or excited, the neural structure is said to be up-regulated; if neural activated is decreased or inhibited, the neural structure is said to be down-regulated. Neural structures are usually assembled in circuits. For example, nuclei and tracts connecting them make up a neural circuit.

Deep Brain Stimulation (DBS) involves implanted electrodes placed within the brain. Typically connecting leads are run down to another part of the body, such as the abdomen where they are connected to the DBS programmer (e.g., Mayberg, H S, Lozano A M, Voon V, McNeely H E, Seminowicz D, Hamani C, Schwalb J M, and S H Kennedy, “Deep brain stimulation for treatment-resistant depression”. Neuron. 45(5):651-60, Mar. 3, 2005).

Transcranial Magnetic Stimulation (TMS) involves electromagnet coils which are powered by brief stimulator pulses (e.g., George M S, Wassermann E M, Williams W, et al., “Changes in mood and hormone levels after rapid-rate transcranial magnetic stimulation of the prefrontal cortex,” J Neuropsychiatry Clin Neuro 1996; 8:172-180; Mishelevich and Schneider, “Trajectory-Based Deep-Brain Stereotactic Transcranial Magnetic Stimulation,” International Application Number PCT/US2007/010262, International Publication Number WO 2007/130308, Nov. 15, 2007).

Ultrasound stimulation is accomplished with focused transducers (e.g., Bystritsky, “Methods for Modifying Electrical Currents in Neuronal Circuits,” U.S. Pat. 7,283,861, Oct. 16, 2007).

Radiosurgery involves permanent change to neural structures by applying focused ionizing radiation in such a way that tissue and thus function are modified but without destroying tissue. A quantity of 60 to 80 grey is typically applied at rates on the order of 5 Gy per minute (e.g., Schneider, Adler, Borchers, “Radiosurgical Neuromodulation Devices, Systems, and Methods for Treatment of Behavioral Disorders by External Application of Ionizing Radiation,” U.S. patent application Ser. No. 12/261,347, Publication No.” US2009/0114849, May 7, 2009).

Transcranial Direct Current Stimulation (tDCS) uses electrode pads external to the scalp that depolarize or hyperpolarize neural membranes on the underlying cortex (e.g., Nitsche, M A, and W. Paulus, “Excitability changes induced in the human motor cortex by weak transcranial direct current stimulation,” J. Physiology, 527.3, 633-639, 2000).

Radio-Frequency (RF) stimulation utilizes RF energy as opposed to ultrasound (e.g., Deisseroth & Schneider, “Device and Method for Non-Invasive Neuromodulation,” U.S. Pat. application Ser. No. 12/263,026, Pub. No.: US2009/0112133. Apr. 30, 2009).

Vagus nerve stimulation involves a programmer in the upper left chest, under the clavicle, with leads wrapped around the vagus nerve with brain stimulation occurring by the vagus connections to brain structures (e.g., George, M., Sackheim, A J, Rush, et al., “Vagus Nerve Stimulation: A New Tool for Brain Research and Therapy,” Biological Psychiatry, 47, 287-295, 2000). Multiple mechanisms have been proposed for the Cyberonics Vagus Nerve Stimulation system for the modulation of mood. These include alteration of norepinephrine release by projections of solitary tract to the locus coeruleus, elevated levels of inhibitory GABA related to vagal stimulation and inhibition of aberrant cortical activity by the reticular activating system (Ghanem T, Early S V, “Vagal nerve stimulator implantation: an otolaryngologist's perspective,” Otolaryngol Head Neck Surg 2006; 135(1):46-51).

Optical stimulation involves methods for stimulating target cells using a photosensitive protein that allows the target cells to be stimulated in response to light (e.g., Zhang, Deisseroth, Mishelevich, and Schneider, “System for Optical Stimulation of Target Cells,” PCT/US2008/050627, International Publication Number WO 2008/089003, Jul. 24, 2008).

Functional stimulation can be accomplished by voluntary movement, induction of sensory input (e.g., pain or pressure) or electrical such as median nerve stimulation (Sailer, Alexandra, G. F. Molnar, D. I. Cunic and Robert Chen, “Effects of peripheral sensory input on cortical inhibition in humans,” Journal of Physiology, 544.2:617-629, 2002).

Drugs can be used for central nervous system effects as well.

### SUMMARY OF THE INVENTION

It is the purpose of this invention to provide methods and systems for non-invasive deep brain or superficial stimulation using multiple modalities simultaneously or on an interleaved basis. This approach is particularly of benefit because impacting multiple points in a neural circuit to produce Long-Term Potentiation (LTP) or Long-Term Depression (LTD). Multiple modalities considered are deep-brain stimulators (DBS) with implanted electrodes, Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation (VNS), functional stimulation, and drugs. Note that VNS is representative of other implanted modalities where nerves located outside the cranium are stimulated and these other implanted modalities are covered by this invention. An example is stimulation of the sphenopalatine ganglion to abort a migraine headache.

### BRIEF DESCRIPTION OF THE DRAWINGS

FIG. 1 shows the characteristics of the various neuromodulation modalities.

FIG. 2 is a table of Indications versus Targets.

FIG. 3 shows a table for Therapeutic-Modality Combinations for Selected Indications.

FIG. 4 shows the physical layout of the combination of therapeutic modalities for the treatment of pain.

FIG. 5 shows the physical layout of the combination of therapeutic modalities for the treatment of depression.

FIG. 6 shows the physical layout of the combination of therapeutic modalities for the treatment of addiction.

FIG. 7 shows the physical layout of the combination of therapeutic modalities for the treatment of obesity.

FIG. 8 shows the physical layout of the combination of therapeutic modalities for the treatment of epilepsy.

FIG. 9 shows a block diagram of the treatment planning and control system.

FIG. 10 illustrates the flow of the treatment planning and control system.

### DETAILED DESCRIPTION OF THE INVENTION

It is the purpose of this invention to provide methods and systems and methods for deep brain or superficial stimulation using multiple therapeutic modalities to impact one or multiple points in a neural circuit to produce Long-Term Potentiation (LTP) or Long-Term Depression (LTD). Some of the modalities (e.g., TMS) will cause training or retraining to bring about long-term change. Radiosurgery (or a surgical ablation) on the other hand will cause a permanent effect and DBS must remain applied or the effect will terminate. Such permanent changes usually will result in down-regulation. Another consideration is that in some cases one does not need a terribly long-term effect such as the application of one or more reversible non-invasive modalities for treatment of an acute condition such as acute pain related to a dental procedure or outpatient surgery.

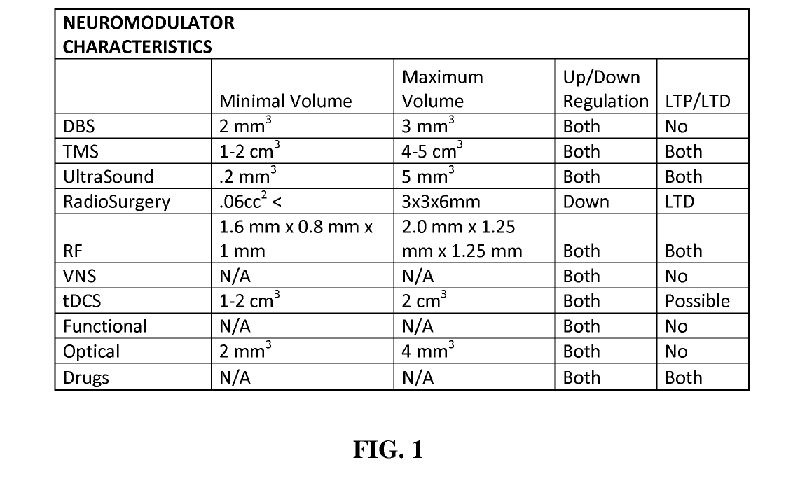

FIG. 1 shows the characteristics of the various neuromodulation modalities. The values for the parameters are approximate and not meant to be absolute. Which treatment modality is to be used in what position for what target depends on such factors as the size of the target (e.g., ultrasound can be focused to 0.5 to 2 mm³ while TMS can be limited to 1-2 cm³ at best), target accessibility, the presence of critical neural structures for which stimulation is to be avoided in proximity to the target, whether side effects will be elicited, local characteristics of the neural tissue (e.g., tDCS can only be used on superficial targets, DBS is not applicable to structures like the Insula that have a high degree of vascularity), whether up or up regulation is to be performed, whether Long-Term Potentiation (LTP) or Long-Term Depression (LTD) is desired, and whether there is physically enough room for the physical combination of neuromodulation elements. Another critical element is whether an invasive modality (e.g., DBS, VNS, optical) is acceptable or not. It is to be noted that radiosurgery can only down-regulate. A fundamental consideration of this invention that a given target may best targeted by one or a set of modalities. For example, a long structure like the DACG may be amenable to deep-brain TMS stimulation while a relatively small target such as the Nucleus Accumbens may be best targeted by DBS. Another consideration is that as the overall clinical therapeutic approach develops, one or more additional modalities may be considered at the point where one or more modalities are already in place. The principles of this invention are important and the invention is not limited to the currently available modalities, because existing techniques will be improved, new techniques will be discovered, and additional targets for given indications will be identified.

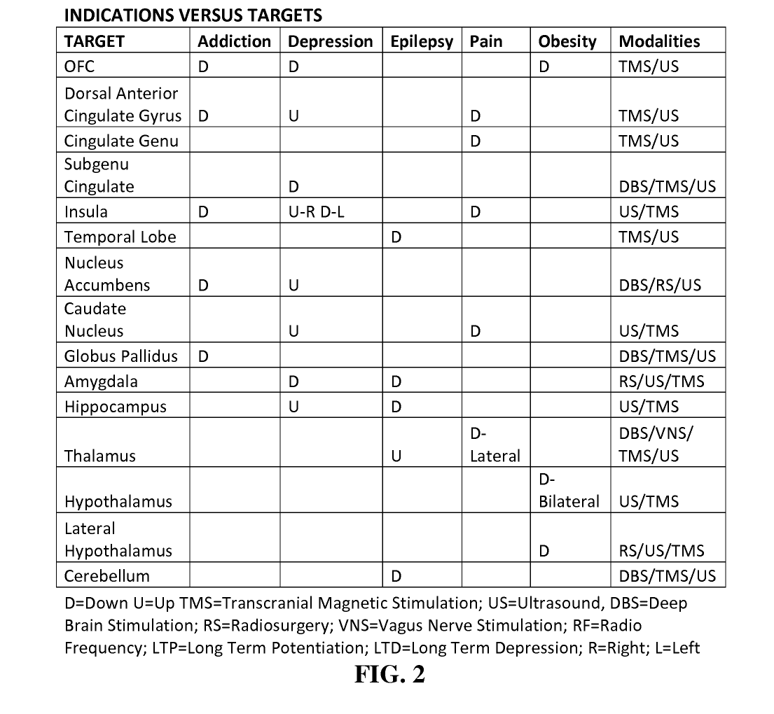

FIG. 2 is a table of Indications versus Targets. Many of these are shown on brainmaps.com. Not all targets for each indication is listed, only the main ones according to current understanding. As additional knowledge is discovered targets or which modality is or modalities are preferable may change. Not all the targets listed need to be hit for treatment to be effective. The entries in each of the indication columns represent either down-regulation (D) or up-regulation (U) for that given target for that indication. Not all targets will be regulated one way or the other for all indications. For example, the Dorsal Anterior Cingulate Gyrus (DACG) is up-regulated for depression and down-regulated for addiction and pain. Likely modalities are listed in the last column of the table. While there may be some preference for the order listed for a given modality according to one judgment the order is by no means mandatory. In some cases, the most effective combination may even be patient specific. In addition, it is possible that other modalities could be used effectively either instead of, or perhaps in addition to a listed modality. Depending on the target set, it may be that using a single modality may also work. An important consideration is that even though many targets are available, in practice one would not necessarily choose to hit all the targets but might well choose a subset. In some cases, there may be too many targets to permit all too be targeted so choices will need to be made. In other cases, it might be possible to set up a combined mechanism to hit all the targets, but it may be too expensive to do so relative to additional benefit to be obtained. In any case, new targets may be discovered as more knowledge is developed.

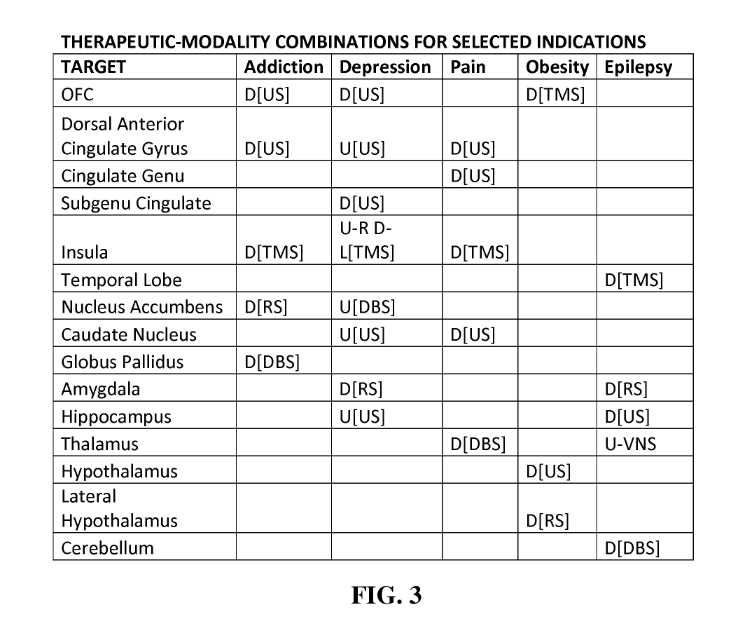

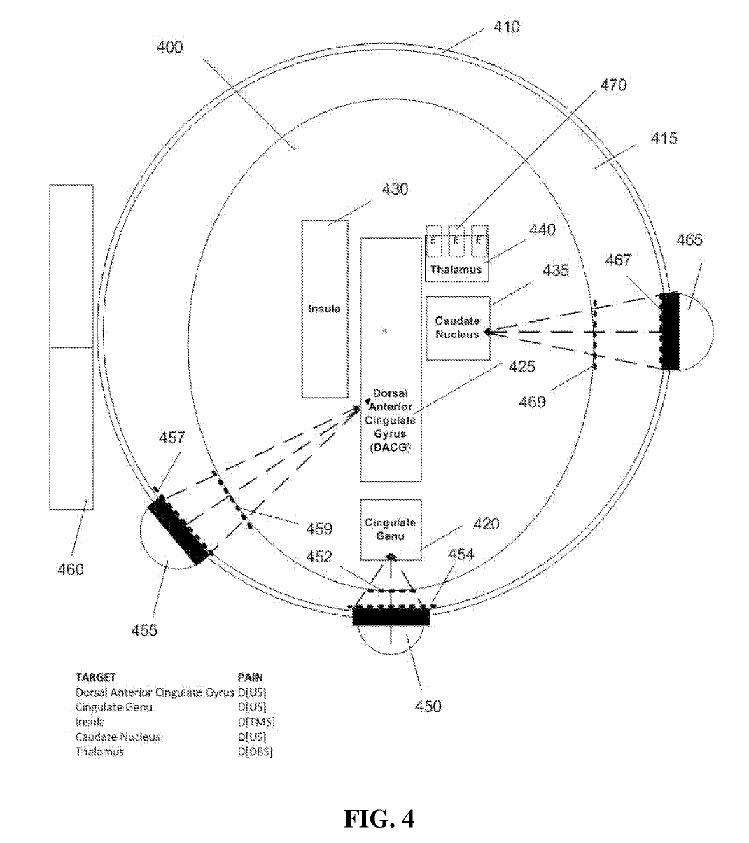

FIG. 3 shows a table for Therapeutic-Modality Combinations for Selected Indications. These represent one combination for each of the five covered indications, pain, depression, addiction, obesity, and epilepsy. The entries in each of the indication columns represent either down-regulation (D) or up-regulation (U) for that given target for that indication plus the particular therapeutic modality to be used. As shown in the diagrams for each seen in FIGS. 4 through 8, an important consideration is the physical space required for each of the energy sources. In some cases moving them off to a different plane and/or orientation may allow tighter packing.

FIG. 4 shows the physical layout of the combination of therapeutic modalities as listed in the table of FIG. 3 for the treatment of pain. The entries from that table just for pain are shown in the lower left-hand corner of the figure for reference. A frame 410 for holding energy sources surrounds head 400. The targets Cingulate Genu 420 neuromodulated by ultrasound transducer 450, Dorsal Anterior Cingulate Gyrus (DACG) 425 neuromodulated by ultrasound transducer 455, Insula 430 neuromodulated by TMS coil 460, Caudate Nucleus 435 neuromodulated by ultrasound source 465, and Thalamus 440 neuromodulated by DBS stimulating electrodes 470 are illustrated. In the case of ultrasonic transducers, the space between frame 410 and head 400 is filled with an ultrasonic conduction medium 415 such as Dermasol from California Medical Innovations with the interfaces between the head and the ultrasonic conduction medium and the ultrasonic medium and the ultrasound transducer are provided by layers of ultrasonic conduction gel, 452 and 454 for ultrasound transducer 450, 457 and 459 for ultrasound transducer 455, and 467 and 469 for ultrasound transducer 465. Note that while specific modalities for the targets are given, appropriate substitutions (i.e., target appropriate to modality, modality physically will fit with the mechanism for the other targets, etc.) can be made. Also, alternative targets to treat a given indication may be appropriate. The preceding points, while included on this section of pain, apply to the indications covered in the following paragraphs and other indications as well. For any of the indications the positions and orientations of the energy sources are set according to the particular needs of the targets and physical configuration. In another embodiment, more than one modality can be used to hit a single target to increase the effect. For example, both ultrasound and TMS could be used to simultaneously or sequentially hit the Dorsal Anterior Cingulate Gyrus.

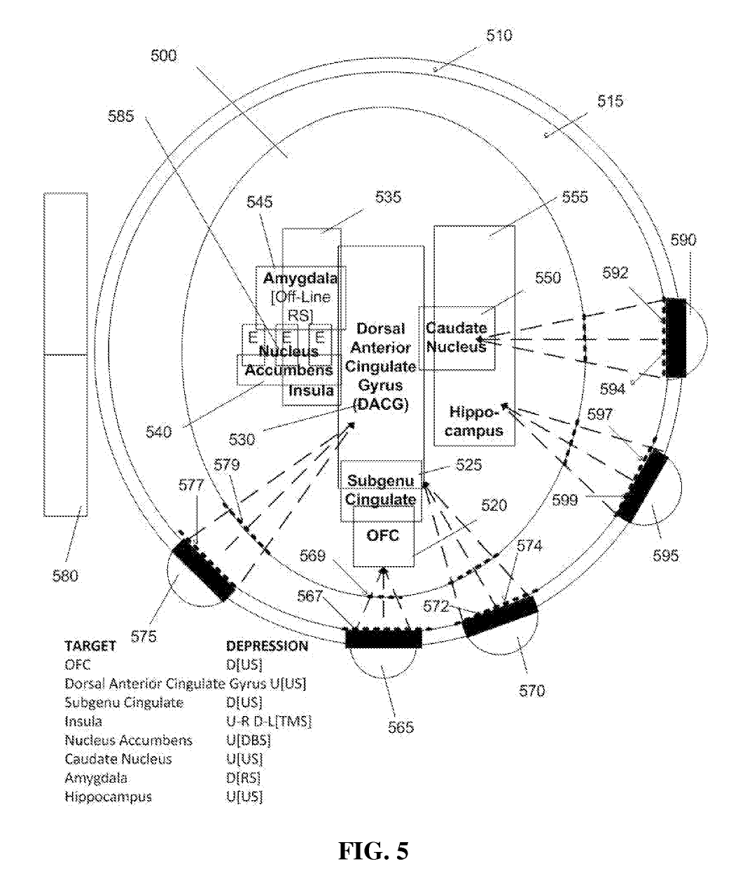

FIG. 5 shows the physical layout of the combination of therapeutic modalities as listed in the table of FIG. 3 for the treatment of depression. The entries from that table just for depression are shown in the lower left-hand corner of the figure for reference. A frame 510 for holding energy sources surrounds head 500. The targets OFC 520 neuromodulated by ultrasound transducer 565, Subgenu Cingulate 525 neuromodulated by ultrasound transducer 570, Dorsal Anterior Cingulate Gyrus (DACG) 530 neuromodulated by ultrasound transducer 575, Insula 535 neuromodulated by TMS coil 580, Nucleus Accumbens 540 neuromodulated by DBS stimulating electrodes 585, Amygdala 545 down-regulated by off-line radiosurgery, Caudate Nucleus 550 neuromodulated by ultrasound source 590, and Hippocampus 555 neuromodulated by ultrasound transducer 595 are illustrated. In the case of ultrasonic transducers, the space between frame 510 and head 500 is filled with an ultrasonic conduction medium 515 such as Dermasol from California Medical Innovations with the interfaces between the head and the ultrasonic conduction medium and the ultrasonic medium and the ultrasound transducer are provided by a layer of ultrasonic conduction gel, 567 and 569 for ultrasound transducer 565, 572 and 574 for ultrasound transducer 570, 577 and 579 for ultrasound transducer 575, and 592 and 594 for ultrasound transducer 590, and 597 and 599 for ultrasound transducer 595. A consideration is that embodiments with alternative configurations (e.g., one or multiple fewer targets) can work as well. It is to be noted that one would expect that additional targets will be discovered as more knowledge is gained so future additions or replacements are expected.

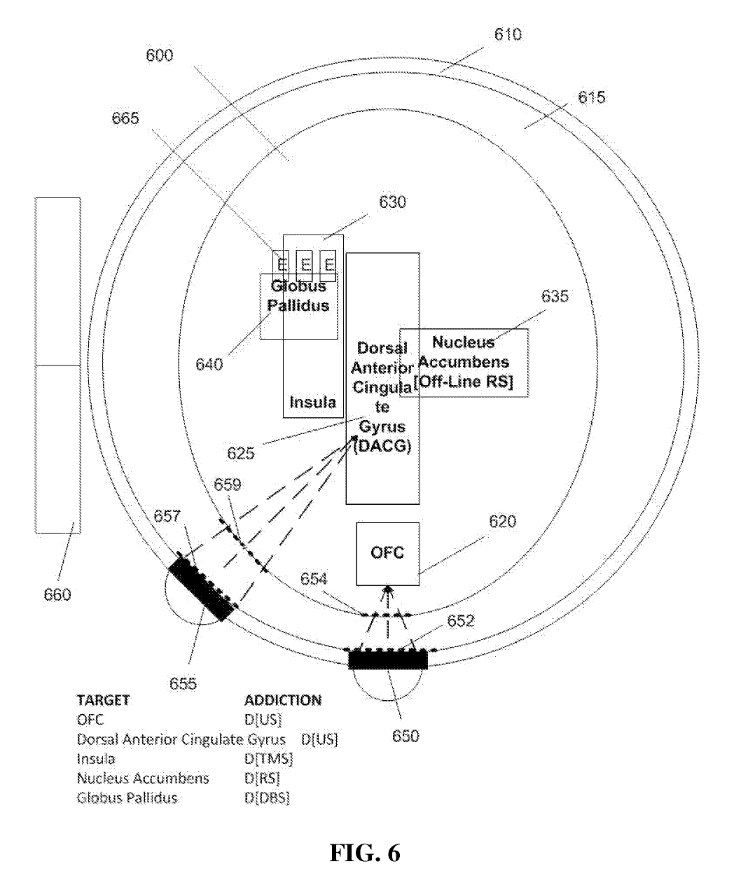

FIG. 6 shows the physical layout of the combination of therapeutic modalities as listed in the table of FIG. 3 for the treatment of addiction. The entries from that table just for addiction are shown in the lower left-hand corner of the figure for reference. A frame 610 for holding energy sources surrounds head 600. The targets OFC 620 neuromodulated by ultrasound transducer 650, Dorsal Anterior Cingulate Gyrus (DACG) 625 neuromodulated by ultrasound transducer 655, Insula 630 neuromodulated by TMS coil 660, Nucleus Accumbens 635 down-regulated by off-line radiosurgery, and Globus Pallidus 640 neuromodulated by DBS stimulating electrodes 665 are illustrated. In the case of ultrasonic transducers, the space between frame 610 and head 600 is filled with an ultrasonic conduction medium 615 such as Dermasol from California Medical Innovations with the interfaces between the head and the ultrasonic conduction medium and the ultrasonic medium and the ultrasound transducer are provided by a layer of ultrasonic conduction gel, 652 and 654 for ultrasound transducer 650, and 657 and 659 for ultrasound transducer 655. Note that in addiction that there are subgroups like smoking vs. drugs for which targets can vary.

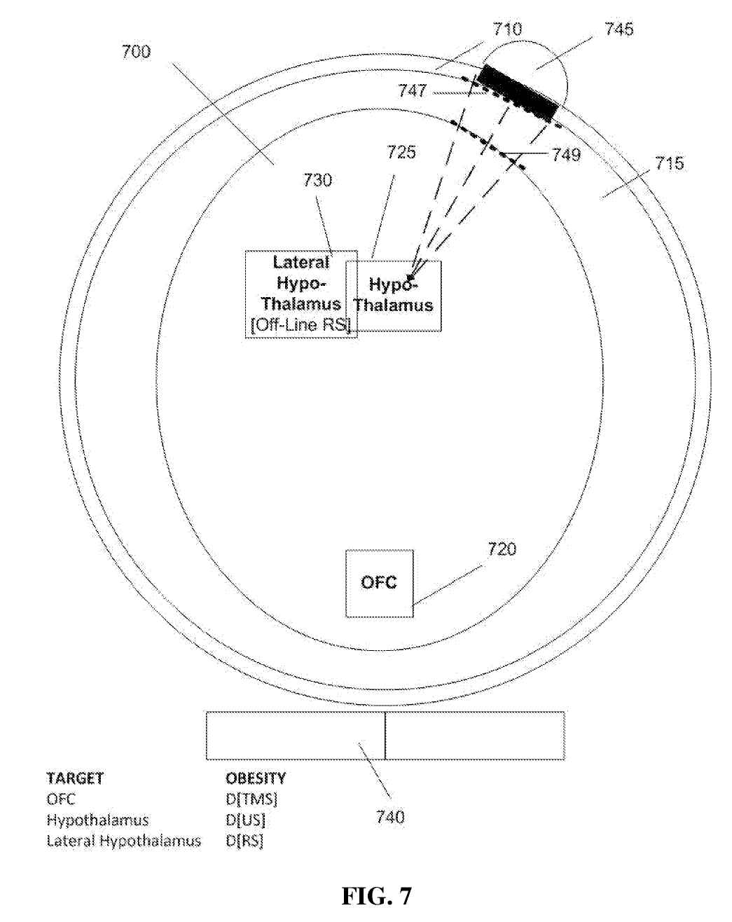

FIG. 7 shows the physical layout of the combination of therapeutic modalities as listed in the table of FIG. 3 for the treatment of obesity. The entries from that table just for obesity are shown in the lower left-hand corner of the figure for reference. A frame 710 for holding energy sources surrounds head 700. The targets OFC 720 neuromodulated by TMS coil 740, Hypothalamus 725 neuromodulated by ultrasound source 745, and Lateral Hypothalamus 730 down-regulated by off-line radiosurgery are illustrated. In the case of ultrasonic transducers, the space between frame 710 and head 700 is filled with an ultrasonic conduction medium 715 such as Dermasol from California Medical Innovations with the interfaces between the head and the ultrasonic conduction medium and the ultrasonic medium and the ultrasound transducer are provided by a layer of ultrasonic conduction gel, 747 and 749 for ultrasound transducer 745.

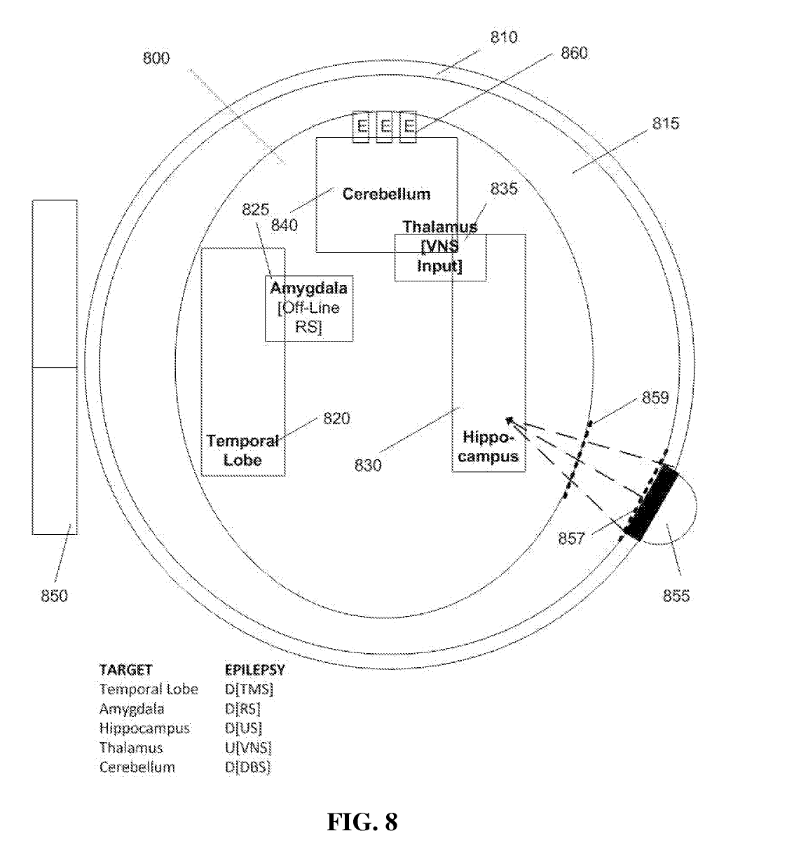

FIG. 8 shows the physical layout of the combination of therapeutic modalities as listed in the table of FIG. 3 for the treatment of epilepsy. The entries from that table just for epilepsy are shown in the lower left-hand corner of the figure for reference. A frame 810 for holding energy sources surrounds head 800. Targets Temporal Lobe 820 neuromodulated by TMS coil 850, Amygdala 825 down-regulated by off-line radiosurgery, Hippocampus 830 neuromodulated by ultrasound source 855, Thalamus 835 neuromodulated by VNS, and Cerebellum 840 neuromodulated by DBS stimulating electrodes 860 are illustrated. In the case of ultrasonic transducers, the space between frame 810 and head 800 is filled with an ultrasonic conduction medium 815 such as Dermasol from California Medical Innovations with the interfaces between the head and the ultrasonic conduction medium and the ultrasonic medium and the ultrasound transducer are provided by a layer of ultrasonic conduction gel, 857 and 859 for ultrasound transducer 855.

Note that where bilateral targets for any indication exist, both sides could be stimulated in other embodiments if the neuromodulation elements can be physically accommodated. Some embodiments may incorporate sequential rather than simultaneous application of on-line, real-time modalities such as ultrasound and TMS. In still other embodiments, multiple indications can be treated simultaneously or sequentially.

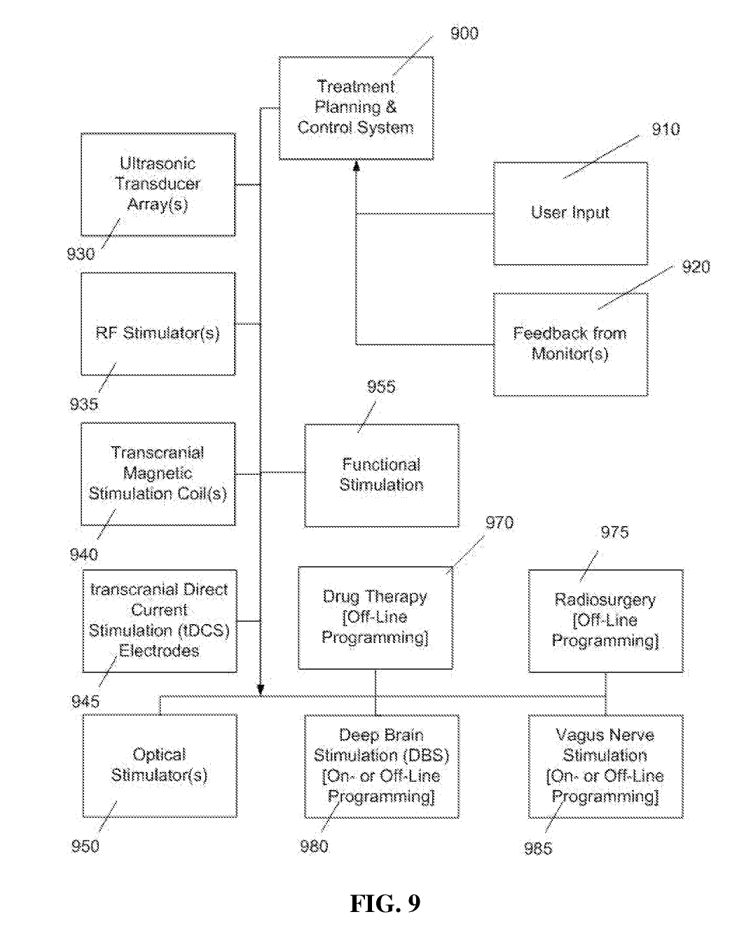

The targeting can be done with one or more of known external landmarks, an atlas-based approach (e.g., Tailarach or other atlas used in neurosurgery) or imaging. The imaging can be done as a one-time set-up or at each session although not using imaging or using it sparingly is a benefit, both functionally and the cost of administering the therapy, over approaches like Bystritsky (U.S. Pat. No. 7,283,861) which teaches consistent concurrent imaging. A block diagram is shown in FIG. 9 that depicts the Treatment Planning and Control System that has inputs from the user and monitoring systems (e.g., energy levels for one or more therapeutic modalities and imaging) and outputs to the various modalities. The treatment planning and control system varies, as applicable, the direction of energy emission, intensity, session duration, frequency, pulse-train duration, phase, firing patterns, numbers of sessions, and relationship to other controlled modalities. Use of ancillary monitoring or imaging to provide feedback is optional. Treatment Planning and Control System 900 receives input from User Input 910 and Feedback from Monitor(s) 920 and provides control output (either real-time or instructions for programming) to Transducer Array(s) 930, RF Stimulator(s) 935, Transcranial Magnetic Stimulation Coil(s) 940, transcranial Direct Current Stimulation (tDCS) Electrodes 945, Optical Simulator(s) 950, Functional Stimulation 955, Drug Therapy 970 [Off-Line Programming], Radiosurgery 975 [Off-Line Programming], Deep Brain Stimulation (DBS) 980 [On- or Off-Line Programming], and Vagus Nerve Stimulation (VNS) 985 [On- or Off-Line Programming] There are four categories of output modalities:
a) on-line-real-time where neuromodulation parameters are changed immediately under direct control of the Treatment Planning and Control System (e.g., ultrasound transducers or TMS stimulators),
b) on-line-prescriptive where neuromodulation parameters are directly set in programmers (e.g., DBS or Vagus Nerve Stimulation programmers) and the effect is both reversible and seen immediately,
c) off-line-prescriptive-adjustable where instructions are generated for users to adjust drug dosages or adjust programmers and the effect is reversible but the effect is seen at a later time after the programmers (e.g., DBS or Vagus Nerve Stimulation programmers) have been so adjusted, and
d) off-line-prescriptive-permanent where neuromodulation parameters are instructions are generated for users to adjust parameters and the effect is not reversible (e.g., radiosurgery) and the effect is seen at a later time after the change has been made.
   Examples of types of control exercised are positioning transducers, controlling pulse frequencies, session durations, numbers of sessions, pulse-train duration, firing patterns, and coordinating firing so that hitting of multiple targets in the neural circuit using firing patterns is done with optimal effects. In addition, in some cases, firing patterns (Mishelevich, D. J. and M. B. Schneider, “Firing Patterns for Deep Brain Transcranial Magnetic Stimulation,” PCT Patent Application PCT/US2008/073751, published as WIPO Patent Application WO/2009/026386) can be used where multiple energy sources of the same or different types are impacting a single target. This strategy can be used to avoid over-stimulating neural tissues between an energy source and the target to avoid undesirable side effects such as seizures. Positioning of neuromodulators and their settings may be patient specific in terms of (a) the actual position(s) of the target(s), (b) the neuromodulation parameters for the targets, and (c) the functional interactions among the targets. In some case performing imaging or other monitoring, may help in determining adjustments to be made, whether those adjustments are made manually or automatically.

In some cases, an off-line procedure will have already been permanently done (e.g., radiosurgery) and for that modality what occurred would only appear as an input. Control will involve such aspects such as the firing patterns that are employed in each of the applicable modalities, the pattern of stimulation among the employed modalities, and whether simultaneous or sequential neuromodulation is employed (including off-line modalities which will automatically mean sequential neuromodulation is done, if any of the therapeutic modalities in the combination are applied in real-time).

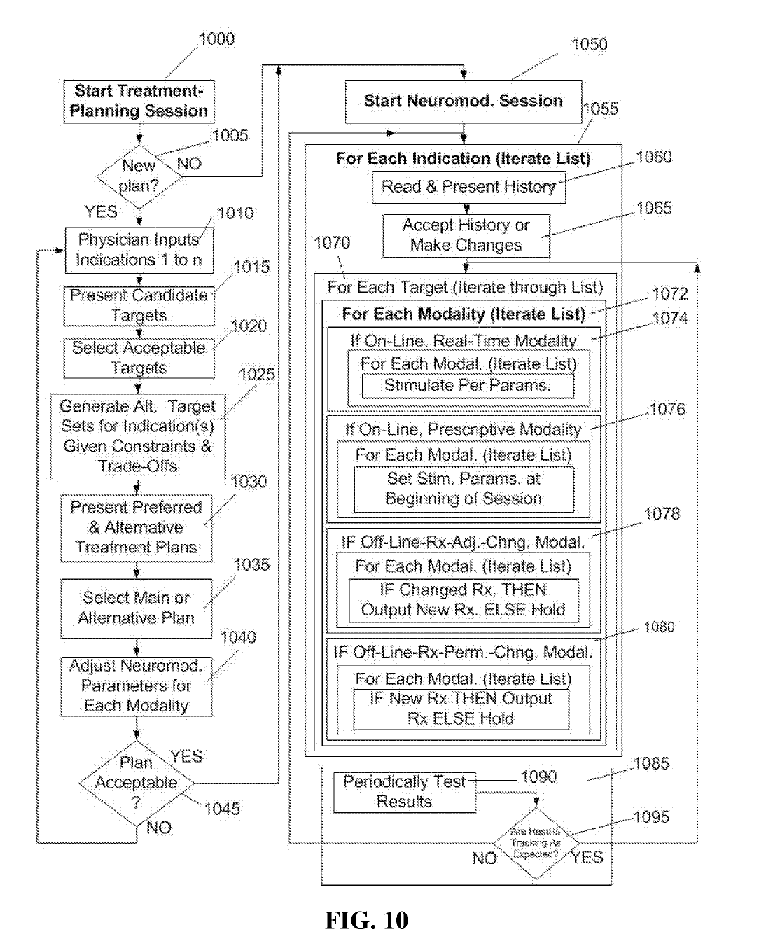

FIG. 10 illustrates the flow for the Treatment Planning and Control System. Just after the start of the Treatment-Planning Session 1000, a branch 1005 occurs which depending on whether this is a new plan (for a new patient) proceeds (if the result is yes) to the physician putting in the indications to be treated 1010 or proceeds (if the result is no) to the start of the Neuromodulation Session 1050.

The flow for the development of the new plan is for in 1010 the physician to input the desired indications followed by the presentation of candidate targets to the physician in 1015. There may be only a single indication. The physician selects the acceptable targets in 1020 and then the system generated alternative target sets associated with the selected indication(s) in 1025 given that physical constraints are satisfied. Trade-offs are given in terms of risk, anticipated relative benefits, possible side effects, and other factors. The resultant preferred treatment plan plus alternative plans are presented to the physician in 1030 and the physician makes the selection of what is to be done in 1035 and adjusts the neuromodulation parameters for each of the modalities in 1040. A branch 1045 follows related to whether the resultant plan is acceptable to the physician. If the answer is no, then the process is repeated with the physician again inputting the desired indications in 1010. If the answer is yes and the results plan is acceptable, then the Neuromodulation Session is started in 1050.

The Neuromodulation Session consists of iterating through each of the designated indications in 1055. For each indication, the system reads and presents the history in 1060 and the physician in 1065 accepts the historical values or makes changes. Then in 1070 the system iterates through each of the designated targets and, then within target, in 1072, the system iterates through each of the appropriate modalities. The actions depend on the category of the modality. If the case involves an On-Line, Real-Time Modality in 1074, the modalities are iterated through, and the given modality is stimulated according to the parameter set. If the case involves an On-Line Prescriptive Modality 1076, then for each of the modalities, the stimulation parameters are set in the given programmer at the beginning of the session. Not all programmers can be automatically set by another system such as the Multi-Modality Treatment-Planning and Control system of the invention, so this mechanism may not be available. In any case if such a modality (e.g., DNS or VNS) can be controlled in this way, the set stimulation will usually continue after the On-Line, Real-Time Modalities such as TMS or Ultrasound session is complete. If the case involves an Off-Line-Prescriptive-Adjustable-Change Modality 1078, then for each of the modalities the stimulation parameters for the programmer are changed if there is new prescription or held if there is not. Finally, if the case involves an Off-Line-Prescriptive-Change Modality, then for each of the modalities if there now is a prescription, the prescription is output; otherwise the prescription is held. There may be more than one such a modality of that type (e.g., two or more radiosurgery modalities), each related to a different target.

An evaluation of the results occurs in 1085. Periodically (either within a neuromodulation session or days, weeks, months, or perhaps even years apart) the functional results are tested in 1090. A branch 1095 is executed related to whether the results are tracking as expected. If the answer is no, then the flow returns to 1055 and each of the indications is iterated through including reading and presenting the history 1060 with physician accepting the historical parameter sets or altering them in 1065 prior to executing the overall program in 1070. If the answer is yes, then no parameter-set changes are required and the flow returns directly to executing the overall program in 1070.

The invention can be applied to a number of conditions including, but not limited to, addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, and epilepsy. In addition it can be applied to cognitive enhancement, hedonic stimulation, enhancement of neural plasticity, improvement in wakefulness, brain mapping, diagnostic applications, and other research functions. In addition to stimulation or depression of individual targets, the invention can be used to globally depress neural activity which can have benefits, for example, in the early treatment of head trauma or other insults to the brain.

A key aspect of the invention is that multiple conditions may be treated at the same time. This can be because the indications to be treated share a single target (e.g., the Dorsal Anterior Cingulate Gyrus (DACG) is down regulated in the treatment of both addiction and pain), or multiple targets in multiple circuit are neuromodulated. The treatment of multiple conditions is likely to become increasingly important as the average age of a given population increases. For example when stroke is being treated, in some cases, it will be practical to treat another condition as well. In treating indications with a common target, one most consider whether that target is neuromodulated in the same direction for both conditions. Otherwise, if for one condition the target is to be up-regulated and for the other condition the target is to be down-regulated, there is a conflict.

All of the embodiments above are capable of and usually would be used for targeting multiple targets either simultaneously or sequentially. Hitting multiple targets in a neural circuit in a treatment session is an important component of fostering a durable effect through Long-Term Potentiation (LTP) and/or Long-Term Depression (LTD). In addition, this approach can decrease the number of treatment sessions required for a demonstrated effect and to sustain a long-term effect. Follow-up tune-up sessions at one or more later times may be required.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What is claimed is:

1. A method of modulating deep-brain targets using multiple therapeutic modalities, the method comprising:
applying a plurality of therapeutic modalities to a deep-brain target,
applying power to each of the on-line therapeutic modalities via a control circuit thereby neuromodulating the activity of the deep brain target regions, and
working in coordination with the off-line therapeutic modalities.

2. The method of claim 1, where the therapeutic modalities are selected from the group consisting of implanted deep-brain stimulation (DBS) using implanted electrodes, Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, drugs.

3. The method of claim 1, wherein a therapy selected from the group consisting of implanted deep-brain stimulation (DBS) using implanted electrodes, Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, functional stimulation, and drugs is combined one or more therapies selected from the group consisting of are implanted deep-brain stimulators (DBS), Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation other-implant stimulation, functional stimulation, drugs.

4. The method of claim 1, wherein a disorder is treated by neuromodulation, the method comprising modulating the activity of one target brain region or simultaneously modulating the activity of two or more target brain regions, wherein the target brain regions are selected from the group consisting of NeoCortex, any of the subregions of the Pre-Frontal Cortex, Orbito-Frontal Cortex (OFC), Cingulate Genu, subregions of the Cingulate Gyrus, Insula, Amygdala, subregions of the Internal Capsule, Nucleus Accumbens, Hippocampus, Temporal Lobes, Globus Pallidus, subregions of the Thalamus, subregions of the Hypothalamus, Cerebellum, Brainstem, Pons, or any of the tracts between the brain targets.

5. The method of claim 1, wherein the disorder treated is selected from the group consisting of: addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, epilepsy.

6. The method of claim 1, wherein the multi-modality therapy is applied for the purpose selected from the group consisting of: cognitive enhancement, hedonic stimulation, enhancement of neural plasticity, improvement in wakefulness, brain mapping, diagnostic applications, and other research functions.

7. The method of claim 1, wherein one or a plurality of targets are hit by a plurality of therapeutic modalities.

8. The method of claim 1, wherein a feedback mechanism is applied, wherein the feedback mechanism is selected from the group consisting of functional Magnetic Resonance Imaging (fMRI), Positive Emission Tomography (PET) imaging, video-electroencephalogram (V-EEG), acoustic monitoring, thermal monitoring.

9. The method of claim 1 where the output is on-line, real time where neuromodulation parameters are changed immediately under direct control of the Treatment Planning and Control System.

10. The method of claim 1 where the on-line, real-time neuromodulators are selected from the group consisting of ultrasound transducers, TMS stimulators.

11. The method of claim 1 where the output is on-line prescriptive where neuromodulation parameters are directly set in programmers and the effect is both reversible and seen immediately.

12. The method of claim 1 where the on-line, prescriptive neuromodulators are selected from the group consisting of on-line, real-time programmable DBS programmers, Vagus Nerve Stimulation programmers, neuromodulators with similar characteristics to DBS programmers, Vagus Nerve Stimulation programmers, other-implant programmers.

13. The method of claim 1 where the output is off-line prescriptive adjustable where instructions are generated for users to adjust programmers and the effect is reversible but the effect is seen at a later time after the programmers have been so adjusted.

14. The method of claim 1 where the off-line, prescriptive adjustable neuromodulators are selected from the group consisting of off-line prescriptive adjustable DBS programmers, Vagus Nerve Stimulation programmers, other-implant programmers, neuromodulators with similar characteristics to DBS programmers, Vagus Nerve Stimulation programmers other-implant programmers.

15. The method of claim 1 where the output is off-line prescriptive permanent where neuromodulation parameters are instructions are generated for users to adjust parameters and the effect is not reversible and the effect is seen at a later time after the change has been made.

16. The method of claim 1 where the off-line, prescriptive permanent neuromodulators are selected from the group consisting of radiosurgery, neuromodulators with characteristics similar to radiosurgery.

17. The method of claim 1 where the treatment planning and control system varies, as applicable, a plurality of elements selected from the group consisting of direction of energy emission, intensity, pulse-train duration, session durations, numbers of sessions, frequency, phase, firing patterns, number of sessions, relationship to other controlled modalities.

18. The method of claim 1 where real-time modalities are applied simultaneously.

19. The method of claim 1 where real-time modalities are applied sequentially.

20. The method of claim 1 where multiple indications are treated simultaneously or sequentially.

21. The method of claim 1 where the multiple conditions have one or more common targets.

22. The method of claim 1 where the multiple conditions have no common targets.

23. A method of modulating deep-brain targets using multiple therapeutic modalities for the treatment of pain, the method comprising:
applying down-regulation via ultrasound to the Dorsal Anterior Cingulate Gyrus,
applying down-regulation via ultrasound to the Cingulate Genu,
applying down-regulation via Transcranial Magnetic Stimulation to the Insula,
applying down-regulation via ultrasound to the Caudate Nucleus, and
applying down-regulation via Deep Brain Stimulation of the Thalamus.

24. The method of claim 23 wherein a therapy selected from the group consisting of implanted deep-brain stimulation (DBS) using implanted electrodes, Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, drugs is replaced by one or more therapies selected from the group consisting of are implanted deep-brain stimulators (DBS), Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, drugs.

25. The method of claim 23 where alternative targets in an applicable neural circuit are substituted.

26. A method of modulating deep-brain targets using multiple therapeutic modalities for the treatment of depression, the method comprising:
applying down-regulation via ultrasound to the Orbito-Frontal Cortex,
applying up-regulation via ultrasound to the Dorsal Anterior Cingulate Gyrus,
applying down-regulation via ultrasound to the Subgenu Cingulate,
applying down-regulation via ultrasound to the Cingulate Genu,
applying up-regulation via Transcranial Magnetic Stimulation to the right Insula,
applying down-regulation via Transcranial Magnetic Stimulation to the left Insula,
applying up-regulation via Deep Brain Stimulation to the Nucleus Accumbens,
applying up-regulation via ultrasound to the Caudate Nucleus,
applying down-regulation via radiosurgery of the Amygdala, and
applying down-regulation via Deep Brain Stimulation of the Thalamus.

27. The method of claim 26 wherein a therapy selected from the group consisting of implanted deep-brain stimulation (DBS) using implanted electrodes, Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, drugs is replaced by one or more therapies selected from the group consisting of are implanted deep-brain stimulators (DBS), Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, drugs.

28. The method of claim 26 where alternative targets in an applicable neural circuit are substituted.

29. A method of modulating deep-brain targets using multiple therapeutic modalities for the treatment of addiction, the method comprising:
applying down-regulation via ultrasound to the Orbito-Frontal Cortex,
applying up-regulation via ultrasound to the Dorsal Anterior Cingulate Gyrus,
applying down-regulation via Transcranial Magnetic Stimulation to the Insula,
applying down-regulation via radiosurgery of the Nucleus Accumbens, and
applying down-regulation via Deep Brain Stimulation of the Globus Pallidus.

30. The method of claim 29 wherein a therapy selected from the group consisting of implanted deep-brain stimulation (DBS) using implanted electrodes, Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, drugs is replaced by one or more therapies selected from the group consisting of are implanted deep-brain stimulators (DBS), Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, drugs.

31. The method of claim 29 where alternative targets in an applicable neural circuit are substituted.

32. A method of modulating deep-brain targets using multiple therapeutic modalities for the treatment of obesity, the method comprising:
applying down-regulation via Transcranial Magnetic Stimulation of the Orbito-Frontal Gyrus,
applying down-regulation via ultrasound to the Hypothalamus,
applying down-regulation via Transcranial Magnetic Stimulation to the Insula, and
applying down-regulation via radiosurgery of the Lateral Hypothalamus.

33. The method of claim 32 wherein a therapy selected from the group consisting of implanted deep-brain stimulation (DBS) using implanted electrodes, Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, drugs is replaced by one or more therapies selected from the group consisting of are implanted deep-brain stimulators (DBS), Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, drugs.

34. The method of claim 32 where alternative targets in an applicable neural circuit are substituted.

35. A method of modulating deep-brain targets using multiple therapeutic modalities for the treatment of epilepsy, the method comprising:
applying down-regulation via Transcranial Magnetic Stimulation of the Temporal Lobe,
applying down-regulation via radiosurgery of the Amygdala,
applying down-regulation via ultrasound to the Hippocampus,
applying up-regulation via Vagus Nerve Stimulation of the Thalamus, and
applying down-regulation via Deep Brain Stimulation of the Cerebellum.

36. The method of claim 35 wherein a therapy selected from the group consisting of implanted deep-brain stimulation (DBS) using implanted electrodes, Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, and drugs is replaced by one or more therapies selected from the group consisting of are implanted deep-brain stimulators (DBS), Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, drugs.

37. The method of claim 35 where alternative targets in an applicable neural circuit are substituted.
